(19) United States
(12) Patent Application Publication
(10) Pub. No.: US 2015/0100042 A1
(43) Pub. Date: Apr. 9, 2015

(54) Muroid Family Nasal Device

(71) Applicant: Impel NeuroPharma Inc., Seattle, WA (US)

(72) Inventors: John D. Hoekman, Seattle, WA (US); Asher Chao-Chi Chiu, Seattle, WA (US); Mei Xu, Seattle, WA (US)

(21) Appl. No.: 14/508,701

(22) Filed: Oct. 7, 2014

Related U.S. Application Data
(60) Provisional application No. 61/887,972, filed on Oct. 7, 2013.

(51) Int. Cl. A61M 25/00; A61M 25/06; A61M 31/00

(52) U.S. Cl. CPC A61M 25/0068; A61M 31/00; A61M 25/0662; A61M 2210/0618; A61M 2250/00
USPC 604/514; 604/239

(57) ABSTRACT

A muroid nasal device for intranasal delivery of compounds to the olfactory region.

### CROSS REFERENCE TO RELATED APPLICATION

This application claims the benefit of U.S. Provisional Application No. 61/887,972 filed Oct. 7, 2013, incorporated herein in its entirety by reference.

### BACKGROUND

The delivery of compounds intranasally to rats and mice for delivery to Central Nervous System (CNS) has been accomplished by the use of intracranial implants. Intracranial implants are an invasive procedure. Depositing drug on the olfactory region of the nasal cavity is difficult to accomplish due to the complex architecture of the nasal cavity and the turbinate guided air path for inhaled breath through the nose. These natural structures act to prevent materials from depositing on the olfactory region as a way to protect this entry way into the CNS. Current nasal drop or spray devices, such as Pfieffer nasal spray devices (Radolfzell, Germany) are designed to saturate the lower nasal cavity. Drug deposited on the lower nasal cavity is absorbed into the blood stream instead of the CNS, eliminating an advantage of using the nasal route for CNS delivery.

A more elegant approach to the delivery of compounds to the CNS of rats and mice is needed.

### SUMMARY

Shown and described is a muroid family nasal device for the delivery of a compound to the muroid family olfactory region.

In one embodiment, the muroid family nasal device shown and described for the intranasal delivery of a compound to an olfactory region of a rat or mouse includes a tube having a distal and a proximal end, the tube having an internal tube diameter and an external tube diameter; a pump connected to the distal end of the tube; a nasal guide having an internal nasal guide diameter greater than the external tube diameter, the tube capable of inserting through the nasal guide with the proximal end of the tube extending from the nasal guide, the nasal guide having about an 18 to about a 25 degree angle or bend inclusive of endpoints. In one aspect, the tube is a catheter. In another aspect, the pump is a syringe.

In one aspect, the device includes a bend in the nasal guide that is 20 degrees.

In another aspect, the device includes a bend in the nasal guide that is 21 degrees.

In another aspect, the device includes a bend in the nasal guide that is 22 degrees.

In yet another aspect, the device includes a bend in the nasal guide that is 23 degrees.

In yet another aspect, the device includes a bend in the nasal guide that is 24 degrees.

In yet another aspect, the device includes a bend in the nasal guide that is 25 degrees.

In one embodiment, a method for delivering a compound to the olfactory region of an animal from the muroid family is describe including providing a nasal guide having a bend of about 18 to about 25 degrees inclusive of endpoints, inserting a tube into the nasal guide, providing a pump to deliver a compound through the tube to the targeted endpoint.

The invention will best be understood by reference to the following detailed description of various embodiments, taken in conjunction with any accompanying drawings. The discussion below is descriptive, illustrative and exemplary and is not to be taken as limiting the scope defined by any appended claims.

### DESCRIPTION OF THE DRAWINGS

The foregoing aspects and many of the advantages will be more readily appreciated as the same become better understood by reference to the following detailed description, when taken in conjunction with the accompanying drawings, wherein:

FIG. 1 is a lateral view of the muroid family nasal device.

FIG. 2 is a top view of the catheter and syringe.

FIG. 3 is a top view of the syringe.

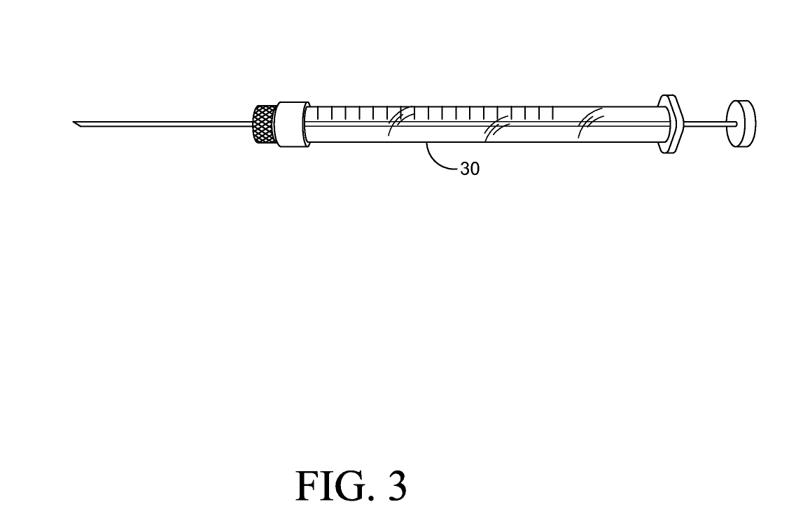

FIGS. 4a and 4b are cross sections of a stained rat nasal cavity showing deposition of a compound using the muroid family nasal device onto the olfactory region. FIG. 4a shows the left half of a rat nasal cavity. FIG. 4b shows the right and left side of a rat nasal cavity. The dark staining identifies the olfactory region.

FIG. 5a shows an arrow indicating the insertion into the rat nasal cavity of a conventional catheter device. FIG. 5b shows an arrow indicating the insertion of a muroid family nasal device into the rat nasal cavity.

### DETAILED DESCRIPTION

When trade names are used herein, applicants intend to independently include the trade name product and formulation, the generic compound, and the active pharmaceutical ingredient(s) of the trade name product.

For clarity of disclosure, and not by way of limitation, the detailed description is divided into the subsections which follow.

Unless defined otherwise, all technical and scientific terms used herein have the same meaning as commonly understood by one of ordinary skill in the art pertinent to the methods, apparatus and compositions described. As used herein, the following terms and phrases have the meanings ascribed to them unless specified otherwise:

“A” or “an” may mean one or more.

To overcome the deficient brain penetration associated with many orally or intravenously administered drugs, the intranasal route is a means to achieve direct drug access to the CNS. The upper region of the nasal cavity provides immediate access to the olfactory epithelium, which, by virtue of being a leaky barrier between the nose and the brain, presents a unique opportunity to deliver drugs into the brain. Drug deposited in this olfactory region results in rapid access to the brain with minimal absorption into the blood. Preclinical studies indicate a rapid transport from the nasal cavity to many regions of the brain and spinal cord at greatly enhanced concentrations compared to systemic drug delivery methods.

Intranasal administration of compounds offers several advantages over traditional surgical, intravenous or oral routes for administration across the blood brain barrier (BBB). Intranasal administration to the olfactory region avoids gastrointestinal destruction and hepatic first pass metabolism, such as destruction of drugs by liver enzymes. Intranasal administration provides ease, convenience and safety. Intranasal drug administration is generally painless and does not require sterile technique, intravenous catheters or other invasive devices, and is generally immediately and readily available for all patients.

Nasally administered compounds which contact the upper olfactory region allow for molecular transport to occur directly across this tissue and into compartments of the central nervous system. (Henry, R. J., et al., Pediatr Dent, 1998. 20(5): p. 321-6; Sakane, T., et al., J Pharm Pharmacol, 1991. 43(6): p. 449-51; Banks, W. A., et al., J Pharmacol Exp Ther, 2004. 309(2): p. 469-75; Westin, et al., Pharm Res, 2006. 23(3): p. 565-72). The olfactory mucosa is located in the upper nasal cavity, just below the cribriform plate of the skull. It contains olfactory cells which traverse the cribriform plate and extend into the cranial cavity. When compounds come in contact with this specialized mucosa, they may be rapidly transported directly into the brain, bypassing the BBB. They may be rapidly transported directly into the central nervous system, often faster than if the compound is given intravenously.

The olfactory mucosa includes the olfactory epithelium. The olfactory epithelium is located at the top of the nose between the superior turbinate and the roof of the nasal cavity, just beneath the cribriform plate of the ethmoid bone. In humans, it covers about 10 to about 20 cm², or about 8% of the total nasal surface area, and is composed of four main cell types: epithelial cells, olfactory receptor neurons, supporting cells, and basal cells. (Mathison S. et al., (1998) Journal of Drug Targeting 5: 415-441). Although 3% of the nasal cavity is occupied by olfactory epithelium (Morrison E E et al, Morphology of the Human Olfactory Epithelium, J Comp Neurol, 297(1):1-13, 1990), this route is direct, since the olfactory neurons do not have a synapse between the receptive element and the afferent path (Ding X and Dahl AR (2003) Chapter 3: Olfactory mucosa: composition, enzymatic localization and metabolism. In Handbook of Olfaction and Gustation, 2nd ed (Doty R L, ed) pp 51-73, Marcel Dekker, New York). The olfactory epithelium is more than twice the depth of the respiratory epithelium, with the olfactory nerve cell bodies typically located in the middle and deeper regions of the epithelium while nuclei of the supporting cells are organized in a single layer closer to the mucosal surface. Tight junctions exist between the supporting cells and between the supporting cells and olfactory nerve cells. (Morrison E. E, et al. (1990) Journal of Comparative Neurology 297(1): 1-13).

When a nasal drug formulation is delivered deep and high enough into the nasal cavity, the olfactory mucosa is reached and drug transport into the brain and/or CSF via the olfactory receptor neurons may occur. The transfer of compounds from the nose to the brain is referred to as the nose-to-brain pathway.

Nasal spray and powder devices have been described in the literature that claim to target the olfactory region. (Charlton et al, International Journal of Pharmaceutics, Volume 338, (June 2007), pg 94). Cannulas or catheters used to deliver compounds to the central nervous system of rats via the animal's nasal cavity have relied upon the skill of the technician to manually position the cannula. For example but not limited to, the skilled technician has to position the cannula posterior to the narrow entrance of the nasoturbinate and maxilloturbinate. (Charlton et al, International Journal of Pharmaceutics, Volume 338, (June 2007), pg 96). Further, the skill of the technician is required so as to deliver the compound and to do so without trauma to the animal, such as but not limited to nasal bleeding. Skilled technicians are not consistently successful. Inconsistent deposition of drug on the olfactory region is an experimental factor limiting the nose to brain distribution route. (Hoekman et al., AAPS PharmSciTech, Vol. 12, No. 2, June 2011, pg. 534). Distribution via nasal drops or catheters tend to involve a large dose volume resulting in saturation of the respiratory epithelium and at the same time olfactory epithelium, limiting their effectiveness for investigation of the olfactory region. (Hoekman et al., AAPS PharmSciTech, Vol. 12, No. 2, June 2011, pg. 535).

Hoekman et al discuss how the administration of 10 μl of dye per naris via conventional nose drops resulted in the dye being localized completely to the respiratory epithelium. (Hoekman et al., AAPS PharmSciTech, Vol. 12, No. 2, June 2011, pg. 538). No noticeable dye staining was apparent in the olfactory region, or in the trachea or esophagus. (Hoekman et al., AAPS PharmSciTech, Vol. 12, No. 2, June 2011, pg. 538). The nose drop administration of 30 μl of dye per naris resulted in saturation of the entire nasal cavity and thus deposition throughout the respiratory epithelium and possibly partially on the olfactory epithelium. (Hoekman et al., AAPS PharmSciTech, Vol. 12, No. 2, June 2011, pg. 538).

Chartlon et al describes dosing of 30 μl per 250 g rat of a simple solution (control) using a straight catheter. (Charlton et al, International Journal of Pharmaceutics, Volume 338, (June 2007), pg 96, Table 1). When dosing 30 μl of a simple solution to the respiratory region of a rat while the rat is on its back, the simple solution quickly drains to the roof and back of the nasal cavity, where the olfactory epithelium is situated. (Charlton et al, International Journal of Pharmaceutics, Volume 338, (June 2007), pg 101). Further, dosing of a rat with a straight catheter results in pooling in the olfactory region of the simple solution formulation due to the supine position of the rat (Charlton et al, International Journal of Pharmaceutics, Volume 338, (June 2007), pg 102). Delivery to the olfactory epithelium in Charlton et al and Hoekman et al is resultant of delivery of 30 μl or greater of dye per naris and dependent upon saturation of the entire nasal cavity. The muroid family nasal device 1 provides for delivery to the olfactory region of 10 μl of compound.

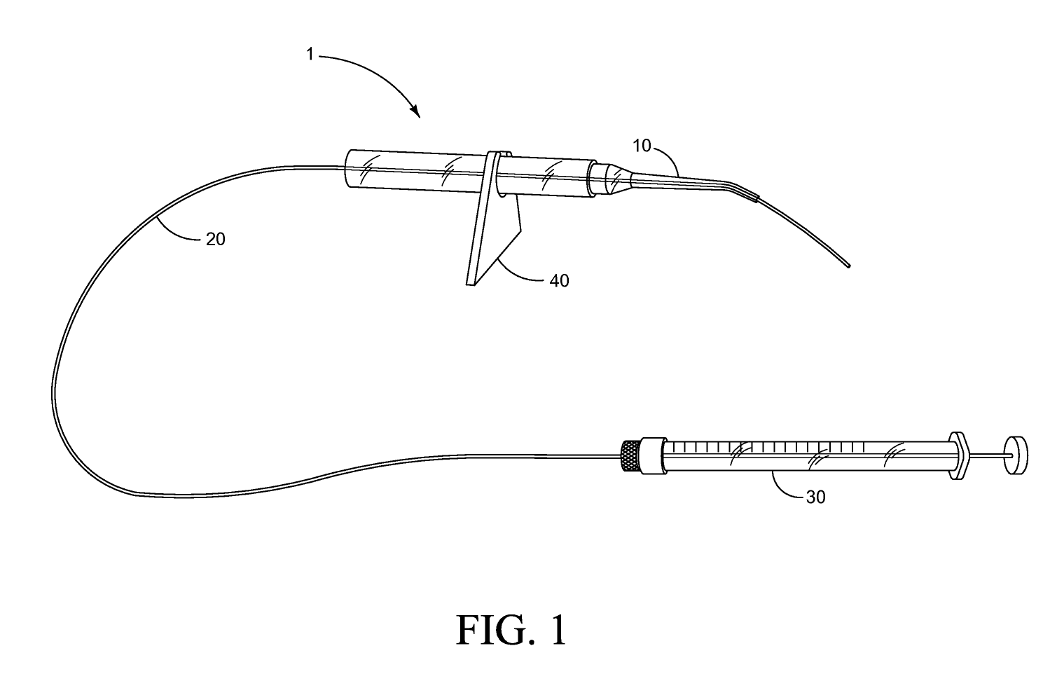

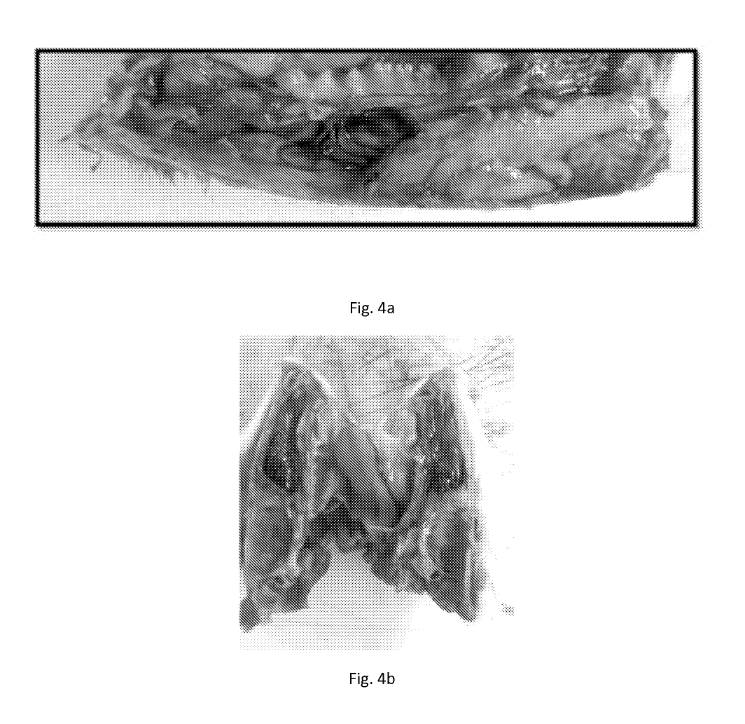

FIG. 1 shows a lateral view of the muroid family nasal device 1. The muroid family nasal device 1 provides for delivery of compounds to the olfactory region of the muroid family nasal cavity, as for example shown in FIGS. 4a and 4b. FIG. 1 shows a nasal guide 10, tube 20 and pump 30. An optional base 40 is shown in FIG. 1. The base 40 may be used to steady the device 1 while in use. Additionally, the base 40 may aid in positioning of the device 1. Additionally, using the base 40 may aid in freeing up the hands of the person using the device 1. The device 1 may be secured to the base 40 via a clamp, grove, magnets or the like.

In one embodiment, the nasal guide 10 is a tube or channel with two openings: one opening at the distal end and one opening at the proximal end. In one aspect, the proximal end of the nasal guide 1 tapers. In another aspect, the nasal guide 10 can be constructed from, for example but not limited to, a 20 uL Raining® pipette tip. As shown in FIG. 1, the nasal guide 10 is bent at about an 18 to about a 25 degree angle, including endpoints, to guide the tube 20 in the direction of the olfactory region. In one aspect, the nasal guide 10 is bent 20 degrees about 5 mm from its proximal end; from the bend to the distal end is about 6 cm. The 6 cm long portion of the nasal guide 10 helps support the tube 20 during insertion. Other lengths of the nasal guide 10 from the bent to the distal end are envisioned which will provide support to the tube 20. Approximately 20 degrees is the angle between the opening of the naris and the roof of the nasal cavity in a rat. This 20 degree bend provides that the tube 20 is traveling in the correct direction while being inserted into the nasal cavity, and that it reaches the target, the olfactory region. In operation, the nasal guide 10 is inserted into the rat naris past the nasal valve.

In yet another aspect, the nasal guide 10 is bent about 18 degrees about 5 mm from its proximal end; in yet another aspect, the nasal guide 10 is bent about 19 degrees about 5 mm from its proximal end, in yet another aspect, the nasal guide 10 is bent about 21 degrees about 5 mm from its proximal end, in yet another aspect, the nasal guide 10 is bent about 22 degrees about 5 mm from its proximal end, in yet another aspect, the nasal guide 10 is bent about 23 degrees about 5 mm from its proximal end, in yet another aspect, the nasal guide 10 is bent about 24 degrees about 5 mm from its proximal end, and in yet another aspect, the nasal guide 10 is bent about 25 degrees about 5 mm from its proximal end.

In yet another aspect, the nasal guide 10 is a tube with a 1 mm outer diameter (OD) and a greater than or equal to 0.7 mm inner diameter (ID) that introduces the tube 20 into the nasal cavity. The inner diameter of greater than or equal to 0.7 mm allows the tube 20 that has a 0.5 mm OD to slide within the nasal guide 10 freely. The outer diameter of 1 mm allows comfortable insertion into the naris of an adult male Sprague Dawley rat. The nasal guide 10 is inserted into the nasal cavity about 5 mm past the opening of the nose. The nasal valve of the rat includes a spiral shaped soft tissue that covers and protects the naris, as well as the cartilage that supports the vestibule of the nose. The proximal end of the nasal guide 10 is inserted into the naris of the rat.

In another aspect, the first 5 mm of the proximal end of the nasal guide 10 does not exceed 1 mm in outer diameter. This allows the inner diameter to be narrow enough to support the tube 20 so it does not bend when it is being slid into the nasal cavity.

FIG. 1 also shows the tube 20 of the muroid family nasal device 1. In one embodiment, the tube 20 can be constructed from a catheter or cannula. In one aspect, the cannula or catheter may be, for example but not limited to, a 30 gauge (0.5 mm OD) light wall tube. In one aspect, the tube 20 is constructed of polytetrafluoroethylene (PTFE). The tube 20 is approximately 20 inches long. It is envisioned that the length of the tube 20 may vary to accomplish delivery from the pump 30 via the tube 20 to the target region. The tube 20 holds the compound as it extends to the targeted region where the compound is to be deposited on the olfactory region. The front end of the tube 20 is inserted about 2 cm into the nasal cavity of an adult male Sprague Dawley rat. The distance between the opening of the nose to the upper olfactory region covered with olfactory epithelium is roughly 2 cm. The length of insertion will vary depending on the age of the rat. The younger and smaller the rat, the shorter the insertion length of the catheter tube will be, vice versa.

The compound to be delivered is contained in a pump 30. For example but not limited to, a pump may consist of a plunger which fits tightly within a cylindrical tube (e.g. a barrel). The plunger can be pushed or pulled inside the cylindrical tube (e.g. barrel) allowing the pump to take in and expel a liquid or gas through an orifice at the end of the barrel. The open end of the pump may be fitted with a needle or element to help direct the flow into and out of the barrel. In one embodiment, the pump 30 is a syringe or syrette. In one aspect, the syringe or syrette is for example, but not limited to, a 25 uL Hamilton® syringe. In operation, the compound to be delivered exits the pump 30, travels along the tube 20, past the nasal guide 10, and exits the muroid family nasal device 1 and is deposited on the olfactory region of the nasal cavity.

In one embodiment, an optional base 40 may be provided. In one aspect, the base 40 is constructed from, for example but not limited to, a metal block and a ring with a screw and is used for holding the device 1 in place once the device 1 is inserted into the rat's nose.

Table 1 describes the distance traveled by the catheter and the angle range for the rat and mouse.

TABLE 1

| | Range | Distance |
|---|---|---|
| Species | of angles | Catheter Travels |
| Rat | 20-25 degrees | 2 cm |
| Mouse | 18-22 degrees | 0.7 cm |

In operation, the tube 20 is designed to travel alongside the roof of the nasal cavity once inserted. In preparation for use, the animal is placed on its back and during use it lies on its back in a supine position. Using a pump 30 to deliver the compound allows the compound to flow slowly onto the olfactory region and saturate the turbinate adjacent to the cribriform plate, as well as the cribriform plate itself as shown in FIGS. 4a and 4b. The darker area shows deposition of the compound being delivered onto the olfactory region.

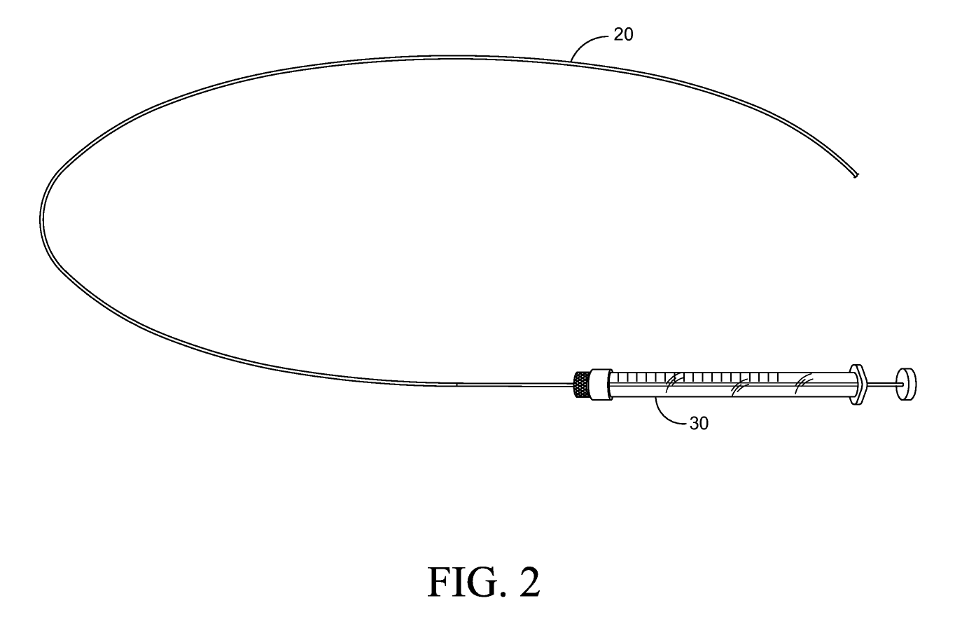

As shown in FIG. 2, the distal end of the tube 20 is connected to a pump 30, in this embodiment, a syringe, by compression fitting the syringe needle into the tube 20. The size of the pump 30 depends on the volume of the dose. For an average dose volume of 10 ul, a 25 ul syringe is used to account for dead space in the tube 20. For example, in one aspect, the pump 30 is a 26-gauge needle (using the Hamilton® company gauge system) and a 25uL Hamilton® syringe. The PTFE material gives the tube 20 the rigidity needed to traverse the nasal cavity, but the flexibility needed to prevent damage to the nasal cavity tissue. The length of insertion is marked on the tube 20 prior to insertion. The compound is prefilled into the tube 20 by drawing the compound through the tube 20 using the pump 30 used for dosing. The tube 20 is moved into the nasal cavity by sliding the tube forward until reaching the designated length.

The muroid family nasal device 1 may be molded of plastic. The muroid family nasal device 1 may be integrally molded or the device 1 may be assembled from parts. In addition to or other than plastic, the muroid family nasal device 1 may contain metal, rubber or glass parts. For example, the materials of which the muroid family nasal device 1 is constructed may be selected to provide sufficient strength and durability to the muroid family nasal device 1 while bearing in mind reducing the leaching of the materials used for construction into the compounds which may be delivered via the muroid family nasal device 1. The muroid family nasal device 1 in experimental animal use may or may not be constructed of medical grade or FDA approved materials. In some aspects, the device 1 may be constructed of but not limited to plastics which are typically polymers of high molecular weight and may contain other substances to improve performance and/or reduce costs. Plastics may include but are not limited to PTFE, polyolefin, PP (polypropylene) or PE (polyethylene), TPE (thermoplastic elastomer) or TPV, TEEE (thermoplastic elastomers with ether and ester groups), and TPU (thermoplastic urethane) or UPE.

### EXAMPLE 1

A straight catheter (as described in Charlton et al, International Journal of Pharmaceutics, Volume 338, Issues 1-2, 29 June 2007, Pages 94-103 and Hoekman et al., AAPS PharmSciTech, Vol. 12, No. 2, June 2011) and a guided rat nasal catheter device were compared. An unexpected result of the comparison was that the guided rat nasal catheter device preferentially deposited compound on the olfactory region.

Materials. A rat nasal catheter device was used including a base, catheter guide (e.g., nasal guide), catheter tube and a Hamilton syringe (250 μL Gastight syringe with a 26 gauge removable needle); a camera (Casio Exilim EX-ZR100); perfusion tools—60 mL of normal saline with 1 mM EDTA, perfusion needle and tubing, infusion pump, hemostats, scissors, tweezers; dissection tools—razor blade, tweezers, hemostats and adult male Sprague Dawley rat(s), approximately 300 g.

### Experimental Setup

A rat was perfused and the head was bisected to show the nasal cavity. The septum was left on to preserve the integrity of one side of the nasal cavity. The catheter tube is filled with blue dye so it is easier to see through the septum.

An adult male Sprague Dawley rat weighing approximately 300 g was sacrificed under anesthesia through cardiac exsanguination using 60 mL of normal saline with 1 mM Ethylenediaminetetraacetic acid (EDTA) and the head was bisected along the sagittal plane with a razor blade to expose the nasal cavity. Isoflurane was used to anesthetize the rat at 5% for 3 minutes prior to the start of the perfusion procedure. The head was dissected so that the septum was left intact on the right side of the nasal cavity to preserve its integrity so that the performance of the catheter can be accurately represented. A muroid family nasal device with a 20-degree bend and a conventional straight catheter were used (Charlton et al, International Journal of Pharmaceutics, Volume 338, Issues 1-2,29 June 2007, Pages 94-103) representative of a conventional nasal catheter used for intranasal drug delivery research. Either is filled with a 1% Mark-it blue dye in water solution for better visualization of the catheter tube inside the nasal cavity. In Example 1, the blue dye assists in showing the position of the catheter tube or device.

The bisected head is positioned so the crown of the head is flat against the working surface. The following steps were followed:

Step 1. Insert the catheter guide 5 nm into the nostril. Step 2. Push the catheter tube gently through the catheter guide. For the guided rat catheter, the catheter tube is inserted 2 cm into the nasal cavity, approximately the distance between the opening of the nostril and the upper olfactory region. The length that the catheter tube travels through the catheter guide is pushed forward slowly until it can no longer travel ward. The catheter guide is adjusted so the shaft of the guide is level with the opening of the nostril. Step 3. Once the catheter tube is determined to have reached the targeted area, the catheter tube is gently pulled back, then the catheter guide is removed from the nasal cavity.

If the catheter tube cannot be pushed forward right after the guide is inserted, the guide and tube are removed and then re-inserted. For an adult rat that weights greater than or equal to 250 g, the catheter tube should travel around 2 cm into the nasal cavity before coming to a stop.

The straight catheter guide is inserted ten times. Steps 1 through 3 are repeated ten times for the guided rat nasal catheter device. A picture is immediately taken to record how the catheter or guided rat nasal catheter device performed after Step 2.

The results of the experimental set up are shown in Table 2.

TABLE 2

| Trial | Bend | Straight |
|---|---|---|
| 1 | + | − |
| 2 | + | − |
| 3 | + | − |
| 4 | − | − |
| 5 | + | − |
| 6 | + | − |
| 7 | + | − |
| 8 | + | − |
| 9 | + | − |
| 10 | + | − |

“+” means the muroid nasal device reached the upper olfactory region through the catheter guide.
“−” means the straight catheter did not reach the upper olfactory region.

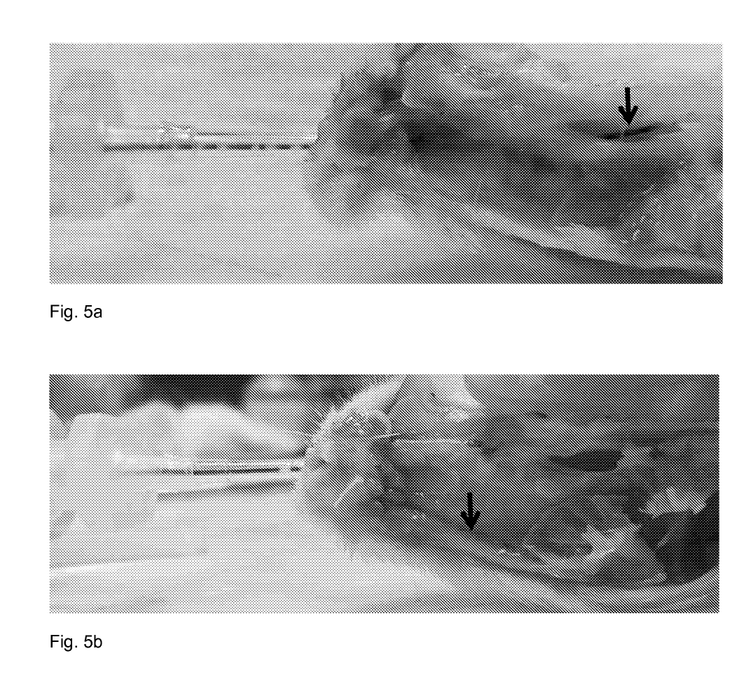

FIG. 5a shows the conventional catheter

(Charlton et al.) inserted into the nasal cavity of a rat. As shown in FIG. 5a and Table 2, the conventional catheter did not reach the upper olfactory region. Notably and not expected, as shown in FIG. 5b and described in Table 2, the rat nasal catheter device reached the upper olfactory region. The respective catheter and device are indicated by the arrow in FIGS. 5a and 5b.

### EXAMPLE 2

An adult male Sprague Dawley rat was anesthetized with 5% isoflurane for three minutes, then 10 uL of 1% Mark-it Blue dye was dosed using the muroid nasal device. The nasal guide was first inserted into the left naris of the animal while under anesthesia to the designated mark, 5 mm from the opening of the naris. The nasal guide was then pushed into the nasal cavity until 2 cm of the tube has been inserted. At this point 10 uL of blue dye was delivered using a Hamilton Syringe. The muroid nasal device was then removed and the animal sacrificed with B-euthanasia (sodium pentobarbital) through cardiac puncture. The nasal cavity of the rat was bisected, exposing the left side of the nasal cavity showing where the dye was deposited. FIG. 4a shows the left side of the rat nasal cavity with dye deposited at the olfactory region. FIG. 4b shows the right and left side of the nasal cavity of the rat with dye deposited at the olfactory region. Unexpectedly, the dye covers the area adjacent to where the tube reached corresponding within the upper olfactory region, about 2 cm from the opening of the nose. This shows that the liquid dose is deposited at the spot where the tube ends up when delivered using a nasal guide and syringe.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

We claim:

1. A device for the intranasal delivery of a compound to an olfactory region of a muroid animal, comprising:
a tube having a distal and a proximal end, the tube having an internal tube diameter and an external tube diameter;
a pump connected to the distal end of the tube;
a nasal guide having an internal nasal guide diameter greater than the external tube diameter, the tube inserted through the nasal guide with the proximal end of the tube extending from the nasal guide, the nasal guide having about an 18 to about a 25 degree bend.

2. The device of claim 1 wherein the bend in the nasal guide is 20 degrees.

3. The device of claim 1 wherein the bend in the nasal guide is 21 degrees.

4. The device of claim 1 wherein the bend in the nasal guide is 22 degrees.

5. The device of claim 1 wherein the bend in the nasal guide is 23 degrees.

6. The device of claim 1 wherein the bend in the nasal guide is 24 degrees.

7. The device of claim 1 wherein the bend in the nasal guide is 25 degrees.

8. The device of claim 1 wherein the pump is a syringe.

9. The device of claim 1 where the tube is a catheter.

10. The device of claim 1 further comprising a proximal end of the nasal guide, wherein the bend in the nasal guide is about 5 mm from the proximal end of the nasal guide.

11. A device for delivering a compound intranasally to an animal of the muroid family comprising:
a nasal guide, the nasal guide having a bend of about 18 to about 25 degrees about 5 mm from a proximal end of the nasal guide, the nasal guide having an opening at the distal and proximal end;
a tube, the tube having a distal and a proximal end, the proximal end of the tube capable of insertion into the opening at the distal end of the nasal guide and exit from the opening at the proximal end of the nasal guide; and
a pump, the pump associated with a distal end of the tube, the pump capable of holding and delivering the compound through the tube.

12. The device of claim 11 wherein the nasal guide is a pipette tip.

13. The device of claim 12 wherein the pump is a syringe.

14. The device of claim 13 wherein the tube extends about 2 centimeters from the proximal end of the nasal guide.

15. The device of claim 14 wherein the tube is a catheter.

16. The device of claim 15 wherein the compound is delivered to the olfactory region of the animal.

17. A method for delivering a compound intranasally to an animal of the muroid family comprising: inserting a nasal guide about 5 mm into a nostril of the animal and delivering a tube through the nasal guide to the olfactory region.

18. The method of claim 17 wherein the tube is inserted about 2 centimeters.

19. The method of claim 17 further comprising removing the tube once reached the olfactory region and then removing the nasal guide from the nostril of the animal.

20. The method of claim 17 further comprising pumping the compound through the tube.
